(19) United States
(12) Patent Application Publication
(10) Pub. No.: US 2015/0174619 A1
(43) Pub. Date: Jun. 25, 2015

(54) System and Method for Cleaning and Retracting at least one Patient Monitoring Cable

(71) Applicant: David Clements, San Francisco, CA (US)

(72) Inventor: David Clements, San Francisco, CA (US)

(21) Appl. No.: 14/313,977

(22) Filed: Jun. 24, 2014

Related U.S. Application Data
(60) Provisional application No. 61/918,265, filed on Dec. 19, 2013.

(51) Int. Cl. B08B 1/02; A61L 2/235; B08B 3/04

(52) U.S. Cl. CPC B08B 1/02; B08B 3/041; A61L 2/235

(57) ABSTRACT

A system and method is provided for cleaning patient monitoring cables including: retracting a cable using a retraction mechanism; receiving, by a housing unit, the cable; and directing, based on movement of the cable, the cable into the housing unit. The innovation may further include: providing cleaning means to the housing unit; protracting the cable; and providing drying means to the housing unit.

### CROSS-REFERENCE TO RELATED APPLICATIONS

The present Utility patent application claims priority benefit of the U.S. provisional application for patent Ser. No. 61/918,265, entitled “Cablemaid”, filed on Dec. 19, 2013 under 35 U.S.C. 119(e). The contents of this related provisional application are incorporated herein by reference for all purposes to the extent that such subject matter is not inconsistent herewith or limiting hereof.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to sanitary care. More particularly, the invention relates to sanitary care of the cables, cords and tubes that run between a patient and the patient monitor in a medical setting and other such objects.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

In some settings, patient monitor cables, cords, and/or tubes which may come into contact with people and/or animals may pick up pathogens and/or other contaminants. In one example, such cables, cords, and/or tubes may touch a floor. In many instances, such cables, cords, and/or tubes may be difficult to clean and/or users may neglect to clean them. An example of this is the cables that connect a patient in a medical setting to the patient monitor that measures their vital signs. These cables can collect pathogens from falling on the floor or from previous patients and if they are not cleaned between patients then pathogens may transfer to the next patient.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. One such aspect shows an electric cord reel designed for mounting adjacent an engine of an automobile that requires a plug-in, heater, and has a power driven retract mechanism operable by an electric switch from an automotive battery or other power source, and which upon completion of retraction will shut off a retracting motor. By way of educational background, another aspect of the prior art generally useful to be aware of teaches of a scalable cable management system comprising an electronics module with a microcontroller, a plurality of drive modules and a releasably engaged cable cartridge for each drive module. Yet another such aspect of the prior art discloses of an ECG lead set assembly including a lead set hub and a retractable reel assembly retained within the lead set hub. However, these solutions may not provide a suitably effective means of retracting and/or cleaning cables, cords, and/or tubes automatically. A solution which did so would be desirable.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is an illustration of an internal view of an exemplary device for retraction, protraction, storage, holding in place, and/or cleaning of various patient monitor cables, cords, and/or tubes, in accordance with an embodiment of the present invention;

FIG. 2 is a drawing of an exemplary device for retraction, protraction, storage, holding in place, and/or cleaning of various patient monitor cables, cords, and/or tubes, in accordance with an embodiment of the present invention; and

FIG. 3 is an illustration of an exemplary method for retraction, protraction, storage, holding in place, and/or cleaning of various patient monitor cables, cords, and/or tubes, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present application or of any further application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

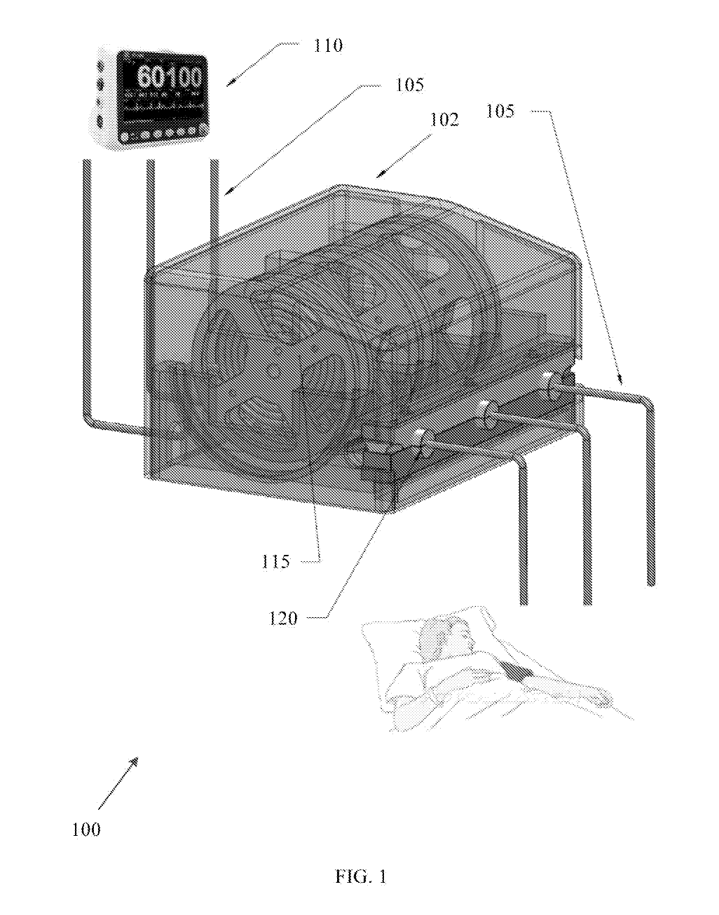

FIG. 1 is an illustration of an internal view of an exemplary device for retraction, protraction, storage, holding in place, and/or cleaning of various patient monitor cables, cords, and/or tubes, in accordance with an embodiment of the present invention. In the present embodiment, device may be suitable for receiving one or more cables 105. In some embodiments, number of cables 105 may be dependent on requirements of a monitor 110 which may be used in conjunction with device. In a non-limiting example, a patient monitor 110 may be designed to receive three cables 105, and a corresponding device may also be suitable for receiving three cables 105. In some embodiments, cables 105 may be monitor cables, such as, without limitation, blood pressure, central line, electrocardiogram (ECG), oxygen saturation, temperature sensing, arterial line, etc. In the present embodiment, device may have a retracting means 115 for recoiling cables 105. In a non-limiting example, a retracting means 115 may recoil cables 105 once cables 105 are disconnected from a patient. It is contemplated that those skilled in the art, in light of the teachings of the present invention, will readily be able to configure patient monitor 110 depending upon the needs (e.g., without limitation, size and portability) of the application. By way of example, and not limitations, in various embodiments, patient monitor 110, could be designed for use in hospital, ambulances, doctors offices, or anywhere the patient may happen to be located when in medical need. In some embodiments, retracting means 115 may be spring-loaded. In many embodiments, device may have one retracting means 115 for each cable 105. In the present embodiment, device may have a cleaning means 120. In some embodiments, cleaning means 120 may be suitable for cleaning and/or disinfecting of cables 105. In a non-limiting example, cleaning means 120 may remove foreign substances and/or apply disinfecting substances. In some embodiments, cleaning means 120 may be composed of any suitable means, including, without limitation, nozzles for spraying cleaning fluid, brushes, a bath of cleaning fluid, and/or foam saturated with cleaning fluid. In a non-limiting example, a retracting cable 105 may rub against saturated foam to remove foreign substances which may have accumulated on cable 105 and/or apply a cleaning fluid. In the present non-limiting example, foam may be disposable and/or replaceable.

In some embodiments, cleaning means 120 may utilize one or more of a variety of cleaning and/or disinfecting agents, including, without limitation, isopropyl alcohol, hydrogen peroxide, quaternary ammonium, ultraviolet light, vapor, etc. In some of these embodiments, cleaning means 120 may also utilize one or more of a variety of cleaning tools, including, without limitation, brushes, nozzles, sponges, foam surfaces, etc.

In many embodiments, a number of retracting means 115 may depend on a number of cables 105. In a non-limiting example, an ICU patient may have multiple cables 105 such as blood pressure, ECG, oxygen saturation, temperature sensing foley line, arterial line, central venous pressure line, etc. In the present non-limiting example, a housing 102 may have six retracting means 115 or two containers each having three retracting means 115 or any number of containers having any number of retracting means 115 to be suitable.

In one or more embodiments, cleaning means 120 may cause cables 105 to become wet during retraction. In some of these embodiments, cables 105 may be allowed to remain wet while inside housing 102. In many instances, cleaning fluid may have evaporating properties which may cause cables 105 to dry quickly when removed from housing 102. In some embodiments, device may have a drying means. In some embodiments, drying means may utilize one or more of a variety of drying tools, including, without limitation, towels, brushes, foam surfaces, rubber blades, etc. In a non-limiting example, a retracting and/or protracting cable 105 may rub against multiple foam surfaces. In the present non-limiting example, one foam surface may be saturated with cleaning fluid and another may be dry so as to effectively absorb cleaning fluid from cables 105. Further, in the present embodiment, a retracting cable 105 may first pass across a dry foam surface followed by a saturated foam surface, then may first pass through saturated surface followed by dry surface when protracting from housing 102 so that cable 105 may be substantially dried before exiting housing 102. If the cables are to be dried as they leave the housing unit, it is most likely to be done using dry foam as addressed above. Most of the liquid cleaning agents need time (a few minutes) to properly kill the pathogens so leaving the cables moist is not a problem. They will dry fairly quickly when exposed to ambient room air.

In some embodiments, device may dispense cleaning agents as needed. In a non-limiting example, device may dispense a cleaning fluid in a metered dose each time a cable 105 retracts. In the present non-limiting example, device may dispense fluid in a variety of ways, such as, without limitation, continually, all at once, in bursts, using a wick technique to repeatedly wick fluid, etc. Some embodiments may utilize any suitable means to deliver cleaning agent to cable 105 and/or cleaning tool, including, without limitation, gravity feed, osmosis, capillary action, siphoning, pumping, etc.

In many embodiments, users may replace various parts of device to achieve a high level of effectiveness. In a non-limiting example, users may replace cleaning foam often to prevent foreign contaminants which foam may have removed while cable was being retracted from being transferred to cables 105 as they are being protracted.

Some embodiments may be suitable for use in transport vehicles. In non-limiting examples, the present device may be used in an ambulance or for medical transport by boat, helicopter or plane.

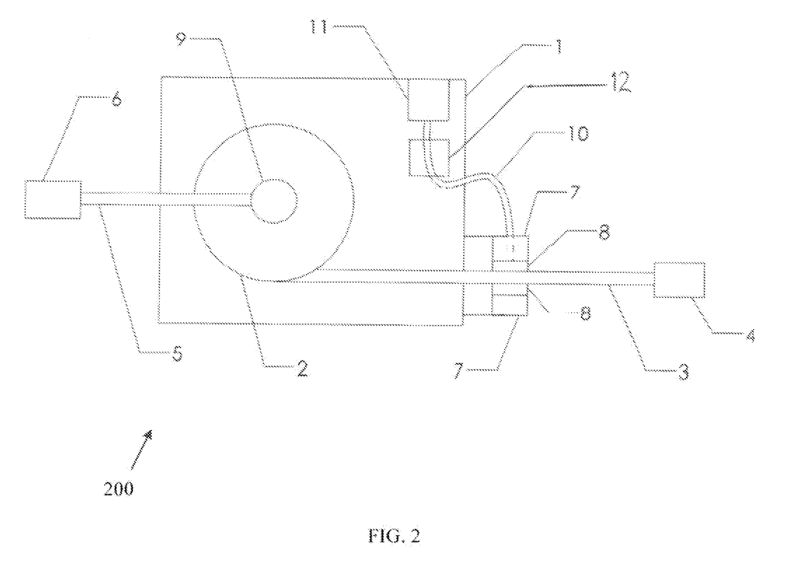

FIG. 2 is a drawing of an exemplary device for retraction, protraction, storage, holding in place, and/or cleaning of various patient monitor cables, cords, and/or tubes, in accordance with an embodiment of the present invention. In the present embodiment, device may have a housing 1, retracting means 2, and one or more cables 3, 5. In some embodiments, device may have multiple cables 3, 5. In the present embodiment, cables 3, 5 may be suitable for extending between a patient 4 and a monitor 6. In some embodiments, a cleaning means 120 may be composed of multiple components. In the present embodiment, a latch 7 may hold a cleaning tool 8 in place. In some embodiments, users may open latch 7 to remove and/or replace cleaning tools 8. In many embodiments, cleaning tool 8 may provide an opening to allow cables 3, 5 to pass through. In a non-limiting example, cleaning tool 8 may be composed of foam and may apply light friction to cable 3, 5 as cable 3, 5 passes through cleaning tool 8. In the present non-limiting example, cleaning tool 8 may allow cleaning agents to flow through and be applied to cable 3, 5 as cable 3, 5 passes through cleaning tool 8. In the present embodiment, a spool shaft 9 may assist in retracting cables 3, 5. Further, in the present embodiment, device may have tubing 10 which may carry cleaning fluid from a fluid reservoir 11 to the cleaning tool 8. In some embodiments, device may not have a fluid reservoir 11. In the present embodiment, device may have a meter 12 which may distribute a predetermined amount of cleaning fluid during retraction of cable 3, 5. In some embodiments, meter 12 may distribute fluid in a steady stream or in bursts.

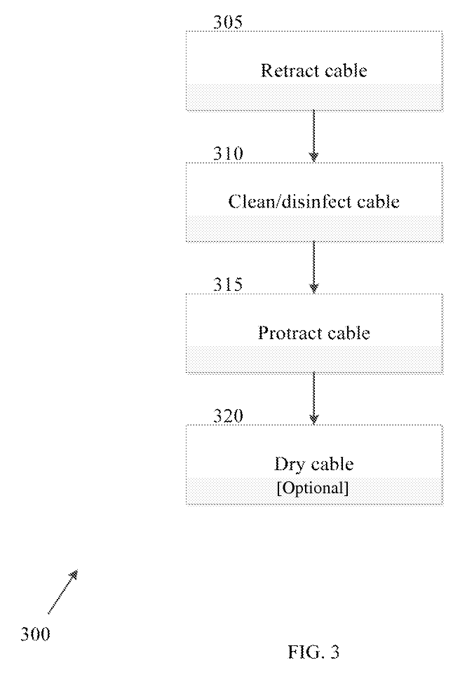

FIG. 3 is an illustration of an exemplary method for retraction, protraction, storage, holding in place, and/or cleaning of various cables, cords, and/or tubes, in accordance with an embodiment of the present invention. In the present embodiment, device may retract a cable 105 in a step 305. In some embodiments, device may use any suitable retracting means 115, including, without limitation, spring-loaded mechanism, motor-driven mechanism, and/or hand-wound mechanism. In a non-limiting example, a device may have a button which a user may push to activate a spring-loaded retracting means 115. In another non-limiting example, a user may pull on a cable 105 to activate a spring-loaded mechanism. In the present embodiment, device may clean and/or disinfect cable 105 in a step 305. In some embodiments, cable 105 may pass through a material such as, without limitation, foam, which may cause friction to remove foreign bodies from cable 105. In some of these embodiments, material may be saturated with a cleaning agent and/or chemicals such as, without limitation, IPA or quantaray ammonia, to kill pathogens on surface of cable 105. In other embodiments, cable 105 may pass through a small bath of cleaning solution, such as, without limitation, IPA or quantaray ammonia. Some embodiments may utilize a variety of other cleaning means 120, including, without limitation, disinfecting vapor, heat, ultraviolet light, pouring a cleaning solution onto cable 105, radiation, dry sterilization process (e.g. hydrogen peroxide), gas and/or liquid sterilizers/cleaners, etc. In many embodiments, device may perform cleaning simultaneously with retraction of cable 105. In other embodiments, device may perform cleaning when cable 105 may be fully within housing 102. In the present embodiment, device may protract cable 105 in a step 315. In some embodiments, device may use any suitable means to protract cable 105, including, without limitation, a motor. In other embodiments, a user may pull on cable 105 to protract it from device. In some embodiments, cleaning means 120 may shift during protraction stage to prevent re-contamination of cable 105 with contaminants on surface of cleaning means 120. In at least one embodiment, device may have a means for alerting users that cleaning means 120 and/or other components may need replacing. In some of these embodiments, suitable alerting means may include, without limitation, a light, alarm, display panel, etc. In a non-limiting example, device may activate alerting means after a predetermined number of cleanings. In another non-limiting example, alerting means may activate after a predetermined amount of time, such as every 24 hours. In some embodiments, alerting means and/or other device components may be powered by solar panels. In the present embodiment, device may dry cable 105 in a step 320.

In some embodiments, device components may be separate structures which may be used together and/or in conjunction with non-embodiment devices. In a non-limiting example, retracting means 115 and cleaning means 120 may be separate devices which may be put together.

By way of example, and not limitation, those skilled in the art, in light of the teachings of the present invention, will readily recognize that the retraction device may be adapted, according to known techniques, with a suitable receptacle (not shown) which receptacle is configured to receive a removable version of the cleaning device, and the receptacle would be designed to properly enable the cleaning device, when properly engaged with said receptacle, to at least clean at least a portion of said first cable while said first cable is being retracted by said retraction device, wherein said cleaning of said first cable performs at least a partially cleaning thereof.

In the present non-limiting example, retracting means 115 and/or cleaning means 1050 may be used in conjunction with a generic patient monitor 110. In another non-limiting example, cleaning means 120 may be used in conjunction with a generic retracting means 115.

In at least one embodiment, retracting means 115 may have a safety feature to prevent cables 105 from retracting too quickly and causing a whipping effect. In other embodiments, retracting means 115 may have slip ring or other suitable means to prevent cables 105 from twisting.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3rd parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing sanitary care according to the present invention will be apparent to those skilled in the art. Various aspects of the invention have been described above by way of illustration, and the specific embodiments disclosed are not intended to limit the invention to the particular forms disclosed. The particular implementation of the sanitary care may vary depending upon the particular context or application. By way of example, and not limitation, the sanitary care described in the foregoing were principally directed to medical care implementations; however, similar techniques may instead be applied to care of any similar objects, such as, without limitation, hoses, pens attached to retractable cords, etc., which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A system comprising:
a retraction device, said retraction device configured to engage and retract, using a suitable retraction mechanism, a first cable, said first cable being configured for suitable usage between a patient monitoring device, said system and a patient; and,
a cleaning device, said cleaning device being configured to at least clean at least a portion of said first cable while (or after) said first cable is being (or was at least partially) retracted by said retraction device, wherein said cleaning of said first cable performs at least a partial cleaning thereof.

2. The system of claim 1, further comprises a protraction mechanism, wherein said protraction mechanism is configured to be operable for protracting the cable.

3. The system of claim 1, wherein said retraction mechanism comprises a retraction mode of operation selected from the group of retraction mechanisms consisting of spring loaded, hand wound, motor driven, engaged by pulling, and engaged by pushing a button.

4. The system of claim 1, further comprises means for protracting the cable.

5. The system of claim 1, wherein said cleaning device further comprises a suitable cleaning agent, wherein said cleaning device is operable to apply said cleaning agent to a portion of the cable.

6. The system of claim 5, wherein said cleaning device further comprises a cleaning agent removal mechanism, wherein said cleaning agent removal mechanism is configured to be operable to at least partially remove the cleaning agent applied to the cable.

7. The system of claim 1, wherein the cleaning means device is further configured to:
identify an amount of the cleaning means inside the first housing unit;
alert a status of the cleaning means according to the amount of the cleaning means inside the first housing unit;
identify an amount of the drying means inside the first housing unit; and
alert a status of the drying means according to the amount of the drying means inside the first housing unit.

8. The system of claim 5, wherein said cleaning agent is a disinfectant soaked foam and said cleaning device is configured to at least partially cover a surface of the first cable

9. The system of claim 6, wherein said cleaning agent removal mechanism comprises a sufficiently dry piece of material, which material is sufficiently dry to be operable for at least partially drying the first cable.

10. A method comprising the Steps of:
retracting, using a retraction mechanism, a first cable; and
cleaning at least a portion of said first cable while said first cable is being retracted into a retraction device, wherein said cleaning of said first cable performs at least a partial cleaning thereof.

11. The method of claim 10, further comprising the Step of protracting the cable.

12. The method of claim 11, further comprising the Step of
providing cleaning means to a first housing unit; and
providing drying means to said first housing unit.

13. The method of claim 10, further comprising:
receiving, by a second housing unit, a second cable;
directing, based on movement of the second cable, the second cable into the second housing unit;
providing cleaning means to the second housing unit; and
providing drying means to the second housing unit.

14. The method of claim 10, further comprising the Steps of
saturating a portion of the first cable inside the first housing unit with the cleaning means; and
drying the saturated portion of the first cable inside the first housing unit with drying means.

15. The method of claim 10, further comprising the Steps of:
identifying an amount of the cleaning means inside the first housing unit;
alerting a status of the cleaning means according to the amount of the cleaning means inside the first housing unit;
identifying an amount of the drying means inside the first housing unit; and
alerting a status of the drying means according to the amount of the drying means inside the first housing unit.

16. The method of claim 14, wherein the step of drying comprises applying a sufficiently dry piece of material capable of at least partially removing at least an applied cleaning agent from the cable

17. The method of claim 14, wherein the step of cleaning further comprises the Step of applying a disinfectant soaked foam upon at least a portion of the cable.

18. An assembly comprising:
means for retracting a cable into a housing;
means for cleaning the cable while the cable is being retracted into said housing by said retraction means; and
means for removing a cleaning agent from said cable, said cleaning agent having been applied by said cable cleaning means.

19. An apparatus comprising:
a retraction device, said retraction device configured to engage and retract, using a suitable retraction mechanism, a first cable, said first cable being configured for suitable usage between a patient monitoring device, said system and a patient; and
a receptacle configured to receive a removable cleaning device, said receptacle being operable for enabling said cleaning device, when properly engaged with said receptacle, to at least clean at least a portion of said first cable while said first cable is being retracted by said retraction device, wherein said cleaning of said first cable performs at least a partial cleaning thereof.

20. The apparatus of claim 19, in which said cleaning device is functionally engaged into said receptacle, and thus said apparatus is operable to perform said cleaning function while the cables are being retracted during normal modes of apparatus operation.
